SEQUENCE LISTING (2 sequences)

SEQ ID NO: 1 - DNA, 1350 bp, Synechococcus
  misc_feature (69)..(70): n is a, c, g, or t
cgtcgcccgctccatgcccgcagctgcgcctttgatgccgcggaagatattgccgccaactaacgatannagtcactgcgatcgcaactaaagccagagatgtgaggaggggatccggcctttggtagactcaactgttggaatccccagaagcaatcatccgtaaggagtcaggacggcgtggagaagacgatcggtctcgagattattgaagttgtcgagcaggcagcgatcgcctcggcccgcctgatgggcaaaggcgaaaagaatgaagccgatcgcgtcgcagtagaagcgatgcgggtgcggatgaaccaagtggaaatgctgggccgcatcgtcatcggtgaaggcgagcgcgacgaagcaccgatgctctatatcggtgaagaagtgggcatctaccgcgatgcagacaagcgggctggcgtaccggctggcaagctggtggaaatcgacatcgccgttgacccctgcgaaggcaccaacctctgcgcctacggtcagcccggctcgatggcagttttggccatctccgagaaaggcggcctgtttgcagctcccgacttctacatgaagaaactggctgcacccccagctgccaaaggcaaagagacatcaataaagtccgcgaccgaaaacctgaaaattctctcggaatgtctcgatcgcgccatcgatgaattggtggtcgtggtcatggatcgtccccgccacaaagagctaatccaagagatccgccaagcgggtgcccgcgtccgtctgatcagcgatggtgacgtttcggccgcgatctcctgcggttttgctggcaccaacacccacgccctgatgggcatcggtgcagctcccgagggtgtgatttcggcagcagcaatgcgttgcctcggcgggcacttccaaggccagctgatctacgacccagaagtggtcaaaaccggcctgatcggtgaaagccgtgagagcaacatcgctcgcctgcaagaaatgggcatcaccgatcccgatcgtgtctacgacgcgaacgaactggcttcgggtcaagaagtgctgtttgcggcttgcggtatcaccccgggcttgctgatggaaggcgtgcgcttcttcaaaggcggcgctcgcacccagagcttggtgatctccagccagtcacggacggctcgcttcgttgacaccgttcacatgttcgacgatgtcaaaacggttagcctgccgttaattcctgatcccaaatggcggccggagcggtagaacgggtatagctcgatcgcttcggtcgttgtttttcagcgaatccatttgcgatcgcttttcaaacccttttttcgtcaaccttctttaaacggcctc

SEQ ID NO: 2 - protein, 356 aa, Synechococcus
VEKTIGLEIIEVVEQAAIASARLMGKGEKNEADRVAVEAMRVRMNQVEMLGRIVIGEGERDEAPMLYIGEEVGIYRDADKRAGVPAGKLVEIDIAVDPCEGTNLCAYGQPGSMAVLAISEKGGLFAAPDFYMKKLAAPPAAKGKETSIKSATENLKILSECLDRAIDELVVVVMDRPRHKELIQEIRQAGARVRLISDGDVSAAISCGFAGTNTHALMGIGAAPEGVISAAAMRCLGGHFQGQLIYDPEVVKTGLIGESRESNIARLQEMGITDPDRVYDANELASGQEVLFAACGITPGLLMEGVRFFKGGARTQSLVISSQSRTARFVDTVHMFDDVKTVSLPLIPDPKWRPER

(19) United States
(12) Patent Application Publication
(10) Pub. No.: US 2016/0010070 A1
(43) Pub. Date: Jan. 14, 2016

(54) METHOD FOR INTRODUCING GENE TO EUGLENA, AND TRANSFORMANT THEREFROM

(71) Applicants: EUGLENA CO., LTD., Minato-ku, Tokyo (JP); KINKI UNIVERSITY, Higashiosaka-shi (JP)

(72) Inventors: Shigeru SHIGEOKA, Nara-shi (JP); Masahiro TAMOI, Nara-shi (JP); Kengo SUZUKI, Minato-ku (JP); Eriko YOSHIDA, Minato-ku (JP)

(73) Assignees: KINKI UNIVERSITY, Higashiosaka-shi, Osaka (JP); EUGLENA CO., LTD., Minato-ku, Tokyo (JP)

(21) Appl. No.: 14/770,715

(22) PCT Filed: Feb. 28, 2014
(86) PCT No.: PCT/JP2014/055161

§ 371 Date: Aug. 26, 2015

(30) Foreign Application Priority Data
Feb. 28, 2013 (JP) 2013-039464
Feb. 28, 2013 (JP) 2013-039465

(51) Int. Cl. C12N 9/16; C12N 15/79

(52) U.S. Cl. CPC C12N 9/16; C12N 15/79; C12Y 301/03011; C12Y 301/03037

(57) ABSTRACT

The present invention provides a method for introducing a gene into Euglena, which can stably maintain a foreign gene, and a transformant therefrom. In this method of introducing a gene into Euglena, a DNA fragment containing an amino acid sequence for encoding a protein is introduced into a Euglena cell. The method includes a step of producing a binary vector containing a DNA fragment, a step of obtaining a linear gene fragment that includes a T-DNA region including the DNA fragment in the binary vector, and a direct gene introduction step of directly introducing the linear gene fragment into the Euglena cell.

### TECHNICAL FIELD

The present invention relates to a method of for introducing a gene to Euglena wherein a foreign gene is introduced to Euglena to cause transformation of the Euglena, and to a transformant of Euglena that exhibits improved yield when cultured.

### BACKGROUND ART

Euglena (generic name: Euglena, Japanese name: Midorimushi) is expected to be used as promising food, fodder, fuel, and the like.

For example, Euglena contains 59 kinds of nutrients such as vitamins, minerals, amino acids, and unsaturated fatty acids, which correspond to a majority of nutrients that are necessary for humans to maintain life, and it has been indicated that Euglena can be used as supplements for enabling well-balanced intake of a variety of nutrients, and as food supply sources in impoverished regions where people cannot take in necessary nutrients.

Further, Euglena is high in protein and nutrition, and hence, it is expected to be used as a fodder for domestic animals and cultured fishes.

Further, Euglena creates oil and fat contents when fixing carbon dioxide by photosynthesis and growing up, and these contents can be utilized as materials for biofuels.

Biofuels are free from worry about exhaustion of resources, unlike fossil fuels such as petroleum. Further, fossil fuels discharges carbon dioxide anew when used as a fuel, but in the case of biofuels, algae, which are plants as raw materials of the biofuels, fix carbon dioxide while growing, and the carbon dioxide is discharged when the biofuels are used as fuels. On the whole, therefore, this does not add an increase in the discharged amount of carbon dioxide, which makes the biofuels be considered effective in preventing global warming.

Still further, in the case of biofuels obtained from edible parts such as corn as raw materials, the use thereof as biofuels and the use thereof as foods conflict, and when such raw materials are used as biofuels, there are fears that food shortage, food price surge, and the like could occur. The use of Euglena, which is not consumed as edible parts at present, however, is free from such conflict with use as foods.

As described above, Euglena is expected to be used as promising food, fodder, and fuel, and has attracted attention for a long time. There are, however, very few examples of successful mass culture of Euglena, for the reasons that Euglena is predated by predators as it is positioned at the lowest bottom of the food chain, and that to set culture conditions such as light, temperature conditions, and the agitate speed is difficult as compared with other microorganisms. Therefore, there is no known example of successful mass culture of Euglena except for the present inventors' success.

Further, in plants for mass culture of rare Euglena, various attempts have been promoted to improve the yield of Euglena as much as possible to stably supply Euglena. For example, transformation of Euglena by gene introduction is expected as one means for achieving an improved yield of Euglena, but no successful example of transformation that allows the yield to improve has been known.

On the other hand, the morphology and the function of a chloroplast significantly vary depending on the differentiation state of an organism that the chloroplast belongs to, and kinds of constituent proteins thereof also vary. The differentiation of chloroplast occurs when various genes encoded in genomes of a nucleus and a chloroplast are subjected to a coordinate and stepwise expression regulation corresponding to a differentiation stage.

Such expression regulation on genes in chloroplast differentiation has hardly been elucidated, and even if a certain gene sequence is successfully introduced to a certain plant, there is very little expectation that the same gene sequence can be introduced to another plant. Further, even if the gene introduction is successful, the introduced gene does not necessarily exhibit a high effect.

Thus, successful examples of gene introduction to higher plants and algae are not necessarily guides that orient the gene introduction into Euglena cells.

In particular, Euglena cells have a unique characteristic that makes researches on the gene introduction difficult. For example, it is known that the amount of DNA of a Euglena cell reaches 50 to 100 times that of algae and molds, which is closer to that of the mammals. It, however, cannot be considered that the entirety of such a large genome is read out and contributes to construction of genes of proteins, and at least it is considered that the genome is formed with many repetitive DNA sequences. In this point, the genome of Euglena is considered unique as a genome of a microorganism.

Further, since Euglena is asexual and does not divide by meiosis, Euglena has been believed to be a genetically extremely stable organism. Though various methods of gene introduction into Euglena have been attempted, there is a feature that not only gene introduction hardly occurs, but also genes are readily eliminated even though introduced. Therefore, there has been no report indicating that a foreign gene was stably maintained in Euglena cells.

One example of an auxotrophic mutant strain derived from a nucleus of Euglena was reported but was not confirmed, and no other mutant strain relating to a gene of a nucleus or mitochondria has been known (Non-patent Document 1). This situation has not changed yet now.

### CITATION LIST

### Non-Patent Literature

NON-PATENT LITERATURE 1: “Euglena, physiology and biochemistry” edited by Shozaburo Kitaoka, Gakkai Shuppan Center Co., Ltd., first edition published on Dec. 10, 1989, page 2

### SUMMARY OF INVENTION

### Technical Problem

The present invention has been made in light of the above-described problems, and an object of the present invention is to provide a method for introducing a gene into Euglena that allows a foreign gene to be stably maintained.

Further, another object of the present invention is to provide a transformant of Euglena that is characterized by improvement of the number of proliferated cells, the cell size, the chlorophyll amount, the photosynthetic activity, and the respiratory activity, and is characterized by improvement of the yield in Euglena culture.

### Solution to Problem

The inventors of the present invention attempted many gene introduction methods and many introduced genes in order to bridge the gap between the prospects of Euglena that is expected as foods, fodders, fuels and the like, and the realization of gene introduction into Euglena that hardly allows mutation to occur and provides a stable gene structure, by solving technical barriers to the realization of gene introduction.

As a result, the inventors successfully realized gene introduction into Euglena, no successful examples of which has been known so far, and at the same time, they found that a foreign gene was stably maintained in Euglena cells. Thus, the inventors completed the present invention.

More specifically, according to the method for introducing a gene into Euglena according to an embodiment, the above-described problem is solved by introducing, to a Euglena cell, a DNA fragment including a base sequence that encodes a protein.

The method may include a step of producing a binary vector containing the DNA fragment, a step of obtaining a linear gene fragment that includes a T-DNA region including the DNA fragment in the binary vector, and a direct gene introduction step of directly introducing the linear gene fragment into the Euglena cell.

In this way, the method includes the direct gene introduction step of directly introducing the linear gene fragment into the Euglena cell, whereby gene introduction into Euglena, which has been believed to be difficult since no successful example was known conventionally, can be achieved.

Here, the direct gene introduction step may include a step of coating microcarriers with the linear gene fragment, and a particle gun step of injecting the microcarriers coated with the linear gene fragment into the Euglena cell by a particle gun method.

In this way, the method includes a particle gun step of injecting the microcarriers coated with the linear gene fragment into the Euglena cells by the particle gun method, whereby stable gene introduction into Euglena, which has been believed to be difficult since no successful example was known conventionally, can be achieved.

Here, the DNA fragment may be a DNA fragment including a base sequence that encodes a protein having activities of fructose-1,6-bisphosphatase/sedoheptulose-1,7-bisphosphatase derived from cyanobacteria.

In this way, a DNA fragment including a base sequence that encodes a protein having activities of fructose-1,6-bisphosphatase/sedoheptulose-1,7-bisphosphatase derived from cyanobacteria is used, which makes it possible to obtain a transformed strain of Euglena, which conventionally has been believed to be very difficult to obtain.

Further, the transformed strain of Euglena obtained is improved regarding the number of proliferated cells, the cell size, the chlorophyll amount, the photosynthetic activity, and the respiratory activity of Euglena.

As a result, the yield of Euglena itself during culture can be improved, and further, yields of useful components such as nutrient components of Euglena, and functions of Euglena, can be improved.

Still further, as the yield and functions of Euglena, mass culture of which is technically difficult, can be improved, it can be expected to open the way for mass supplying of Euglena with view to the use of Euglena as foods, fodders, fuels and the like.

Here, the microcarriers may be microparticles of gold having a diameter of 0.26 μm or less.

When the diameter of microcarriers is 0.26 μm or less, gene fragments are introduced most stably.

Here, the number of proliferated cells, the cell size, the chlorophyll amount, the photosynthetic activity, and the respiratory activity of the Euglena may be improved.

Here, the Euglena may be Euglena gracilis.

With this configuration, a transformant of Euglena suitable for foods, fodders, fuels and the like can be obtained.

Here, the above-described base sequence may encode a protein having an amino acid sequence indicated in (a) or (b) below:

(a) an amino acid sequence indicated by amino acid numbers 1 to 356, in the amino acid sequence represented by SEQ ID NO. 2 in the sequence listing;

(b) an amino acid sequence that is identical to the amino acid sequence of (a) except that a part thereof is deleted, substituted or added, the amino acid sequence exhibiting activities of fructose-1,6-bisphosphatase/sedoheptulose-1,7-bisphosphatase.

Here, the base sequence may be a base sequence indicated in (c) or (d) below:

(c) abase sequence indicated by base numbers 181 to 1251, in the base sequence represented by SEQ ID NO. 1 in the sequence listing;

(d) abase sequence that is identical to the base sequence of (c) except that a part thereof is deleted, substituted, or added, the base sequence encoding a protein that exhibits activities of fructose-1,6-bisphosphatase/sedoheptulose-1,7-bisphosphatase.

With this configuration, a transformed strain of Euglena, which conventionally has been believed to be very difficult, can be obtained.

On the other hand, the inventors of the present invention attempted many gene introduction methods and many introduced genes in order to bridge the gap between the prospects of Euglena that is expected as foods, fodders, fuels and the like, and the realization of gene introduction into Euglena that hardly allows mutation to occur and provides a stable gene structure, by solving technical barriers to the realization of gene introduction. Consequently, the inventors discovered that a gene that encodes a protein having activities of fructose-1,6-bisphosphatase/sedoheptulose-1,7-bisphosphatase derived from cyanobacteria can be introduced to a cell of Euglena, and at the same time, the yield of the transformant obtained is improved. Thus, the inventors completed the present invention.

More specifically, according to a transformant of Euglena according to an embodiment, the above-described problem is solved by a transformant of Euglena obtained by introducing, into Euglena, a gene that encodes a protein having activities of fructose-1,6-bisphosphatase/sedoheptulose-1,7-bisphosphatase derived from cyanobacteria.

As a transformant of Euglena is configured in this manner, the transformant of Euglena that is characterized by improvement regarding the number of proliferated cells, the cell size, the chlorophyll amount, the photosynthetic activity, and the respiratory activity can be obtained.

As a result, the yield of Euglena itself during culture can be improved, and further, yields of useful components such as nutrient components of Euglena, and functions of Euglena, can be improved.

Further, as the yield and functions of Euglena, mass culture of which is technically difficult, can be improved, it can be expected to open the way for mass supplying of Euglena with view to the use of Euglena as foods, fodders, fuels and the like.

Still further, a gene that encodes a protein having activities of fructose-1,6-bisphosphatase/sedoheptulose-1,7-bisphosphatase derived from cyanobacteria is introduced, which makes it possible to obtain a transformant of Euglena, which conventionally has been believed to be very difficult.

Here, the Euglena may be Euglena gracilis.

With this configuration, a transformant of Euglena suitable for foods, fodders, fuels and the like can be obtained.

Further, the gene may be a gene that encodes a protein of (a) or (b) below:

(a) a protein having a 1st to 356th amino acid sequence in the amino acid sequence represented by SEQ ID NO. 2;
(b) a protein that includes an amino acid sequence that is obtained by substituting, deleting, inserting, and/or adding one or more amino acids with respect to a 1st to 356th amino acid sequence in the amino acid sequence represented by SEQ ID NO. 2, and that has activities of fructose-1,6-bisphosphatase/sedoheptulose-1,7-bisphosphatase.

Further, the gene may include a base sequence of (c) or (d) below:

(c) a 181st to 1251st base sequence in the base sequence represented by SEQ ID NO. 1;
(d) a base sequence that includes a base sequence that is obtained by substituting, deleting, inserting, and/or adding one or more bases with respect to a 181st to 1251st base sequence in the base sequence represented by SEQ ID NO. 1, and that encodes a protein that has activities of fructose-1,6-bisphosphatase/sedoheptulose-1,7-bisphosphatase.

This configuration makes it possible to obtain a transformant of Euglena, which conventionally has been believed to be very difficult to obtain.

Further, the gene may be introduced to a nuclear genome and/or a chloroplast genome of the Euglena.

### Advantageous Effects of Invention

According to the present invention, a direct gene introduction step of directly introducing a linear gene fragment to a cell of Euglena is provided, and this makes it possible to achieve stable gene introduction into Euglena, which has been believed to be difficult since no successful example was known conventionally.

Further, in a cell of Euglena, a DNA fragment that includes a base sequence that encodes a protein having activities of fructose-1,6-bisphosphatase/sedoheptulose-1,7-bisphosphatase derived from cyanobacteria is used. This makes it possible to obtain a transformed strain of Euglena, which conventionally has been believed to be very difficult.

Still further, the obtained transformed strain of Euglena is characterized by improvement regarding the number of proliferated cells, the cell size, the chlorophyll amount, the photosynthetic activity, and the respiratory activity of Euglena.

As a result, the yield of Euglena itself during culture can be improved, and further, yields of useful components such as nutrient components of Euglena, and functions of Euglena, can be improved.

Further, as the yield and functions of Euglena, mass culture of which is technically difficult, can be improved, it can be expected to open the way for mass supplying of Euglena with view to the use of Euglena as foods, fodders, fuels and the like.

Further, according to the present invention, a transformant of Euglena that is characterized by improvement regarding the number of proliferated cells, the cell size, the chlorophyll amount, the photosynthetic activity, and the respiratory activity can be obtained.

Still further, a gene that encodes a protein having activities of fructose-1,6-bisphosphatase/sedoheptulose-1,7-bisphosphatase derived from cyanobacteria is introduced, which makes it possible to obtain a transformant of Euglena, which conventionally has been believed to be very difficult.

### BRIEF DESCRIPTION OF DRAWINGS

FIG. 1 is a schematic diagram illustrating a DNA fragment of a linear gene fragment for introduction into Euglena.

FIG. 2 illustrates confirmation of genes to be introduced into Euglena and expressed proteins.

FIG. 3 illustrates the numbers of proliferated cells when a transformed strain and a wild type of Euglena were cultured.

FIG. 4 illustrates photographs of appearances of the transformed strain and the wild type of Euglena at day 8 of culture.

FIG. 5 illustrates chlorophyll amounts of the transformed strain and the wild type of Euglena.

FIG. 6 illustrates photosynthetic activity and respiratory activity of the transformed strain and the wild type of Euglena.

FIG. 7 illustrates the numbers of proliferated cells when the transformed strain and the wild type of Euglena were cultured.

FIG. 8 is graph for comparison between carbohydrate contents in culture solutions of the wild strain and the transformed strain.

FIG. 9 is a photograph for comparison of mucilage accumulation states when the transformed strain and the wild type of Euglena were cultured.

### DESCRIPTION OF EMBODIMENTS

The inventors of the present invention attempted many gene introduction methods and many introduced genes in order to bridge the gap between the prospects of Euglena that is expected as foods, fodders, fuels and the like, and the realization of gene introduction into Euglena that hardly allows mutation to occur and provides a stable gene structure, by solving technical barriers to the realization of gene introduction.

As a result, the inventors discovered the following: in the case where a circular plasmid vector is used, it is very difficult to introduce a gene into Euglena by any of various gene introduction methods such as the Agrobacterium method or the electroporation method, and even if a gene is introduced into Euglena, only a transient expression is achieved; on the other hand, in the case where a linear gene fragment is used, surprisingly, gene introduction is successfully achieved by the direct gene introduction method, and at the same time, a foreign gene is maintained stably in Euglena cells. Thus, the inventors completed the present invention.

Hereinafter, a method for introducing a gene into Euglena and a transformant of Euglena according to one embodiment of the present invention are described in details.

A method for introducing a gene into Euglena according to the present embodiment is characterized by injecting, into cells of Euglena, microcarriers coated with a linear gene fragment including a base sequence that encodes a protein having activities of fructose-1,6-bisphosphatase/sedoheptulose-1,7-bisphosphatase derived from cyanobacteria by the particle gun method, thereby introducing the base sequence into Euglena.

Further, the transformant of Euglena according to the present embodiment is obtained by introducing, into Euglena, a gene that encodes a protein having activities of fructose-1,6-bisphosphatase/sedoheptulose-1,7-bisphosphatase derived from cyanobacteria.

(Euglena to which a Gene is to be Introduced)

Euglena that can be used in the present embodiment is widely distributed in fresh water in ponds and marshes, and Euglena separated from these may be used, or alternatively, arbitrary Euglena that is already isolated may be used.

Examples of Euglena to which a gene is to be introduced by the method according to the present embodiment include the following species categorized in the genus Euglena: Euglena gracilis; Euglena gracilis Klebs; and Euglena gracilis var. bacillaris. Among these, particularly, Euglena gracilis Z strain, SM-ZK strain as a mutant strain of Euglena gracilis Z strain (chloroplast-lacking strain), and var. bacillaris as a variety thereof are used preferably. Alternatively, a gene mutation strain such as chloroplast mutant strains of these species or the like may be used.

### (Linear Gene Fragment Used in Gene Introduction)

For gene introduction according to the present embodiment, a linear gene fragment that includes a gene to be introduced is used, wherein the linear gene fragment is composed of either a DNA fragment that encodes a protein having activities of fructose-1,6-bisphosphatase/sedoheptulose-1,7-bisphosphatase derived from cyanobacteria, or a DNA fragment that has the homology to the above-described DNA fragment.

As the DNA fragment that encodes a protein having activities of fructose-1,6-bisphosphatase/sedoheptulose-1,7-bisphosphatase derived from cyanobacteria, for example, a gene that encodes fructose-1,6-bisphosphatase (hereinafter referred to as FBPase)/sedoheptulose-1,7-bisphosphatase (hereinafter referred to as SBPase) isolated from cyanobacteria Synechococcus PCC 7942 can be used.

### (Protein Having FBPase/SBPase Activities)

The cyanobacteria-derived FBPase/SBPase used in the present embodiment is a protein that can work as a rate-limiting enzyme in the Calvin cycle.

Examples of the protein exhibiting FBPase/SBPase activities include an amino acid sequence of fructose-1,6-bisphosphatase/sedoheptulose-1,7-bisphosphatase derived from cyanobacteria Synechococcus PCC 7942 gene, represented by SEQ ID NO. 2.

This protein is an enzyme that is widely distributed in cyanobacteria, which is a prokaryotic alga, and has a primary structure and enzymological properties that are different from those of FBPase and SBPase of higher plant chloroplasts. Further, the protein is a bifunctional enzyme that has activities of two enzymes of FBPase and SBPase though it is a single protein.

Examples of the protein having FBPase/SBPase activities include a protein composed of the amino acid sequence represented by SEQ ID NO. 2. In the amino acid sequence represented by SEQ ID NO. 2, a 1st to 356th amino acid sequence is the part that has FBPase/SBPase activities; therefore, the protein may include the 1st to 356th amino acid sequence.

Examples of the protein having FBPase/SBPase activities include a protein having an amino acid sequence that is configured so that, in the 1st to 356th amino acid sequence in the amino acid sequence represented by SEQ ID NO. 2, one or more amino acids are deleted, substituted, added or inserted.

Examples of the protein having FBPase/SBPase activities include a protein that has a homology of at least 60%, preferably 80% or more, more preferably 90% or more, and further preferably 95% or more, to the 1st to 356th amino acid sequence in the amino acid sequence represented by SEQ ID NO. 2, and that has FBPase/SBPase activities.

In the present description, when “homology” of an amino acid sequence is mentioned, primary structures of proteins are compared, and this term is used as indicating a level of homology between amino acid residues composing the respective sequences.

When “deleting, substituting, adding, or inserting one or several (about 2 to 6) amino acids” is described regarding an amino acid sequence, this means that deletion, substitution, addition, or insertion of amino acids the number of which is at a natural level is caused by a well-known technique such as the site-directed mutagenesis method.

### (Gene Introduction Method)

A transformant of Euglena according to the present embodiment is produced by introducing, into Euglena, a gene that encodes a protein having activities of FBPase/SBPase derived from cyanobacteria.

Hereinafter, a gene introduction method for producing a transformant of Euglena according to the present embodiment is described.

The gene introduction is performed by to injecting, to a Euglena cell, microcarriers coated with a linear gene fragment that includes a gene that encodes a protein having activities of FBPase/SBPase derived from cyanobacteria by the particle gun method.

### (Gene to be Introduced)

Examples of the DNA fragment including a base sequence that encodes a protein having FBPase/SBPase activities used in the present embodiment include, for example, a gene composed of a base sequence represented by SEQ ID NO. 1. Since the structural gene part that expresses an enzyme is a 181st to 1251st base sequence in the base sequence represented by SEQ ID NO. 1, the DNA fragment may include this part of the base sequence.

Examples of the DNA fragment that encodes a protein having the FBPase/SBPase activities, used in the present embodiment, include a DNA that has a base sequence identical to the DNA sequence represented by SEQ ID NO. 1 described above except that one or several bases are deleted, substituted, added, or inserted, and that encode a protein having FBPase/SBPase activities. When “one or several bases are deleted, substituted, added, or inserted” is mentioned regarding a base sequence, this means that deletion, substitution, addition, or insertion of bases the number of which is at a natural level (one to several bases) is caused by a well-known technique such as the site-directed mutagenesis method.

Examples of the DNA fragment that encodes a protein having FBPase/SBPase activities, used in the present embodiment, includes a DNA that can be hybridized with a DNA composed of base sequences respectively complementary to the DNA sequence represented by SEQ ID NO. 1 under stringent conditions, and that is composed of a base sequence that encodes a protein having FBPase/SBPase activities.

The term “DNA that can be hybridized under stringent conditions” means a DNA that can be obtained by using the above-described DNA as a probe and by using the colony hybridization method, the plaque hybridization method, the southern blott hybridization method, or the like. The term “stringent conditions” means hybridization conditions with a SSC solution having a salt concentration of about a 0.1 to 2-fold concentration (a composition of a SSC solution having a 1-fold concentration consists of 150 mM of sodium chloride and 15 mM of sodium citrate) at a temperature of about 65° C.

Further, examples of the DNA fragment that encodes a protein having FBPase/SBPase activities used in the present embodiment include a DNA that has homology of at least 60% to the DNA sequence represented by SEQ ID NO. 1, and that is composed of a base sequence that encodes a protein having FBPase/SBPase activities.

The term “DNA having homology” means a DNA having homology of at least about 60%, more preferably about 80% or more, more preferably about 90% or more, and further preferably about 95% or more under high-stringent conditions.

The term “high-stringent conditions” means, for example, the following conditions: a sodium concentration of about 19 to 40 mM, more preferably about 19 to 20 mM; and a temperature of about 50 to 70° C., more preferably about 60 to 65° C. Particularly, such conditions as a sodium concentration of about 19 mM and a temperature of about 65° C. are the most preferable conditions.

### (Linear Gene Fragment)

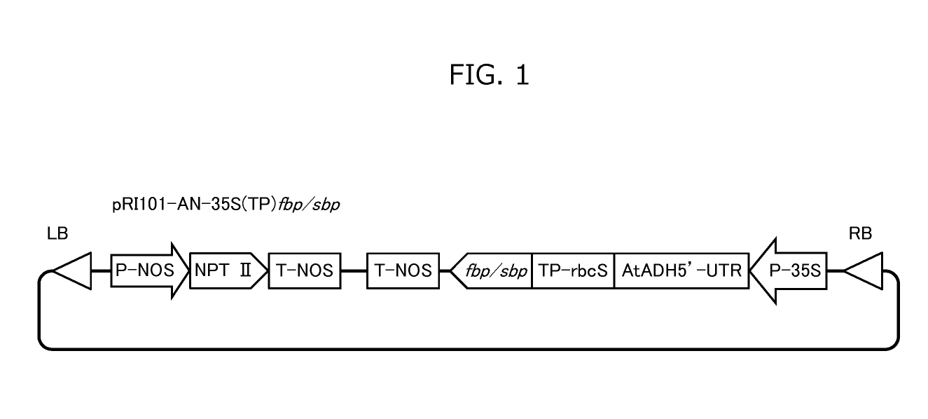

The linear gene fragment used in gene introduction in the present embodiment is illustrated in the schematic diagram in FIG. 1 as one example, and includes an expression cassette of a DNA fragment that encodes a protein having activities of FBPase/SBPase derived from cyanobacteria.

The expression cassette preferably has a chloroplast transit peptide in the upstream of the translation start site of a gene that encodes a protein having activities of FBPase/SBPase derived from cyanobacteria.

As the chloroplast transit peptide, rbcS-TP, which is a transit peptide derived from ribulose-1,5-bisphosphate carboxylase small subunit (RbcS) of a plant, may be used.

The expression cassette preferably further has a translation enhancer region, which is a sequence that promotes translation, in the upstream of the chloroplast transit peptide.

As the translation enhancer region, for example, a 5′ untranslated region (5′-UTR) derived from an ADH (Alcohol Dehydrogenase) gene can be used preferably.

The expression cassette preferably further has a promoter for gene expression in a plant, in the upstream of the translation enhancer region.

The promoter may be adjacent to the translation enhancer region, or may be in the about 1 to 30 base upstream of the same, as long as the promoter is in the upstream of the translation enhancer region.

As the promoter, for example, the following promoter can be used preferably: promoter of elongation factor 1α gene (EF1α promoter); 35S promoter; psbA promoter; PPDK promoter; PsPAL1 promoter; PAL promoter; UBIZM1 ubiquitin promoter; and rrn promoter. Among these, the 35S promoter of cauliflower mosaic virus (CaMV), or the like, can be used particularly preferably.

Further, the linear gene fragment preferably includes a selection marker gene for identifying a genetically modified organism. The selection marker gene is not limited particularly, and a known one can be used.

Examples of such a gene include various types of drug resistance genes (aadA), and genes that complement the auxotrophy of a host. More specifically, the examples include an ampicillin resistance gene, a neomycin resistance gene (G418 resistance), a chloramphenicol resistance gene, a kanamycin resistance gene, a spectinomycin resistance gene, and a URA3 gene. Particularly, a kanamycin resistance gene (NPTII gene (kanr gene)) that expresses a APH(3′)II(NPTII) protein that inactivates aminoglycoside antibiotics is used preferably.

Further, a promoter for recognizing the gene and a terminator of the gene are preferably arranged in the upstream and downstream of the gene, respectively. As the promoter and the terminator, the above-described plant-derived promoter and terminator can be preferably used, and a NOS promoter (P-NOS) and a NOS terminator (T-NOS) derived from a nopaline synthase (NOS) gene of soil bacterium Agrobacterium tumefaciens (Agrobacterium) are particularly preferable.

The linear gene fragment of the present embodiment is prepared by treating a binary vector that includes a sequence of this linear gene fragment with a restriction enzyme to obtain only a T-DNA region interposed between LB and RB (left and right borders).

In the present description, the term “linear gene fragment” means a DNA fragment having a free 5′ terminal and a free 3′ terminal, which is not a circular DNA. Further, the real shape of the linear gene fragment is not necessarily linear, but may have curve or twist. Regarding the morphology of the linear gene fragment upon gene introduction in the present embodiment, the fragment may be double-stranded or single-stranded, but it is preferably double-stranded.

In the case where a circular plasmid vector is used, it is difficult to introduce a gene into Euglena even by any of various gene introduction methods such as the Agrobacterium method or the electroporation method, and even if a gene is introduced into Euglena, only a transient expression is achieved; on the other hand, in the case where a linear gene fragment is used, surprisingly, gene introduction is successfully achieved by the direct gene introduction method, and at the same time, a foreign gene is maintained stably in Euglena cells.

### (Procedure of Gene Introduction)

The method for introducing a gene into Euglena is performed through the following procedure.

First, preculture of Euglena is performed.

Next, gene introduction for introducing the above-described linear gene fragment into Euglena is performed by the known particle gun method.

The gene introduction can be performed by using any of known particle gun devices of the shotgun type, the arc discharge type, the nitrogen gas pressure type, the air gun type, and the helium type; among these, the helium-type device, which directly sprays helium gas to DNA-coated particles provided in a cartridge and injects the particles, is used preferably.

Further, the gene introduction executed in the present embodiment is not limited to the gene introduction by the particle gun method, but may be executed by any method as long as the method enables direct introduction of the above-described linear gene fragment. In particular, the method of directly introducing a foreign gene to cells by applying a mechanical force, which is called the “direct gene introduction method”, is preferably used; for example, the electroporation method, the microinjection method, the PEG method (polyethylene glycol method), or the like can be used.

The T-DNA region obtained from the binary vector, interposed between LB and RB, is amplified by PCR, whereby a linear gene fragment is obtained.

Microcarriers formed with metal microparticles made of gold, tungsten, or the like are coated with the amplified linear gene fragment by a known dry particle method, and gene introduction is performed by the particle gun method using the helium-type device.

After introduction, static culture is performed for 24 hours, and the medium is exchanged with a CM medium containing an antibiotic corresponding to a selection marker gene included in the linear gene fragment, which is followed by selection for several weeks.

An antibiotic resistant strain, appearing, is suspended in a CM medium, and is plated on a selection medium. Colonies appearing one week after are subjected to streak culture, and are further subjected to selection. Using the obtained antibiotic resistant strain, the PCR and the Western blotting analysis are performed.

### EXAMPLE

Hereinafter, the present invention is described more specifically by way of examples and test examples. The present invention, however, is not limited to these.

### Example 1

### Gene Introduction into Euglena

Gene introduction into Euglena was performed by the method described above in the description of the embodiment.

First, preculture of Euglena was performed.

Euglena was cultured for five days in a Koren-Hutner culture medium (hereinafter referred to as a “KH medium”, arginine hydrochloride: 0.5 g/L, aspartic acid: 0.3 g/L, glucose: 12 g/L, glutamic acid: 4 g/L, glycine: 0.3 g/L, histidine hydrochloride: 0.05 g/L, malic acid: 6.5 g/L, citric acid 3Na: 0.5 g/L, succinic acid 2Na: 0.1 g/L, (NH₄)₂SO₄: 0.5 g/L, NH₄HCO₃: 0.25 g/L, KH₂PO₄: 0.25 g/L, MgCO₃: 0.6 g/L, CaCO₃: 0.12 g/L, EDTA-Na₂: 50 mg/L, FeSO₄(NH₄)₂SO₄.6H₂O: 50 mg/L, MnSO₄.H₂O: 18 mg/L, ZnSO₄.7H₂O: 25 mg/L, (NH₄)₆Mo₇O₂₄.4H₂O: 4 mg/L, CuSO₄: 1.2 mg/L, NH₄VO₃: 0.5 mg/L, CoSO₄.7H₂O: 0.5 mg/L, H₃BO₃: 0.6 mg/L, NiSO₄.6H₂O: 0.5 mg/L, vitamin B₁: 2.5 mg/L, vitamin B₁₂: 0.005 mg/L (pH3.5)) under the conditions of continuous irradiation, or for four days in the KH medium under the conditions of light shielding, and thereafter, it was cultured for one day under the conditions of continuous irradiation. A collected culture solution was centrifuged at 3,000 rpm, at room temperature, for ten minutes. Sterilized water was added to the collected precipitates of Euglena so that the precipitates were cleaned, and centrifuge was performed at 3,000 rpm for 10 minutes. Thereafter, 2 mL of a sample containing cells at a cell concentration of 2×10⁷ cells/mL was sucked under a reduced pressure so that the cells were adsorbed to a membrane filter (produced by Millipore Corporation), and this membrane filter was placed on a Cramer-Myers agar medium (hereinafter referred to as a CM agar medium, agarose: 1 g/L, (NH₄)₂HPO₄: 1.0 g/L, KH₂PO₄: 1.0 g/L, MgSO₄.7H₂O: 0.2 g/L, CaCl₂.2H₂O: 0.02 g/L, citric acid 3Na.2H₂O: 0.8 g/L, Fe₂(SO₂)₃.7H₂O: 3 mg/L, MnCl₂.4H₂O: 1.8 mg/L, CoSO₄.7H₂O: 1.5 mg/L, ZnSO₄.7H₂O: 0.4 mg/L, Na₂MoO₄.2H₂O: 0.2 mg/L, CuSO₄.5H₂O: 0.02 g/L, thiamine hydrochloride (vitamin B₁): 0.1 mg/L, cyanocobalamin (vitamin B₁₂): 0.0005 mg/L (pH3.5)) in a petri dish that was covered with an aluminum foil for light shielding, and was subjected to static culture for 24 hours in a culture chamber.

Further, a linear gene fragment to be introduced into Euglena was prepared.

A gene that encodes a protein having FBPase/SBPase activities (fbp/sbp, a gene including a base sequence represented by SEQ ID NO. 1 cloned by the inventors of the present invention) and an antibiotic resistance gene (NPTII) were inserted in a multicloning site of a binary vector pRI101-35S for plants (produced by Takara Bio Inc.), and a chloroplast transit peptide rbcS-TP (acquired from Mr. Sugita in Nagoya University) was inserted in the upstream of the translation start site thereof, whereby a binary vector pRI101-AN-35S(TP) fbp/sbp including T-NDA illustrated in FIG. 1 was produced.

Next, a region interposed between LB and RB illustrated in FIG. 1 was amplified by PCR, whereby a linear gene fragment is obtained.

Gold microparticles were coated with the amplified linear gene fragment by a known dry particle method. As the gold microparticles, those having a diameter of 0.26 μm were used.

Thereafter, by using a known helium-type device, the gold particles were injected by the particle gun method (pressure: 900 psi, distance: 9 cm) into Euglena precultured on a membrane, so that linear gene fragments were introduced into the Euglena cells. Further, the cells were cultured under the conditions of light shielding for one day, and thereafter, the cells were transferred to a CM agar medium containing antibiotic kanamycin (50 μg/ml) and replaced every two weeks, and selection of transformants was carried out. Kanamycin is an antibiotic corresponding to a kanamycin resistance gene (NPTII gene) of a selection marker contained in the linear gene fragment.

A variety of gene introduction conditions have been attempted so far, but gene introduction was successfully performed only by the above-described method. As a result of confirmation of expression by the PCR and the Western blotting method using the obtained antibiotic resistant strain, it was clarified that Euglena was transformed.

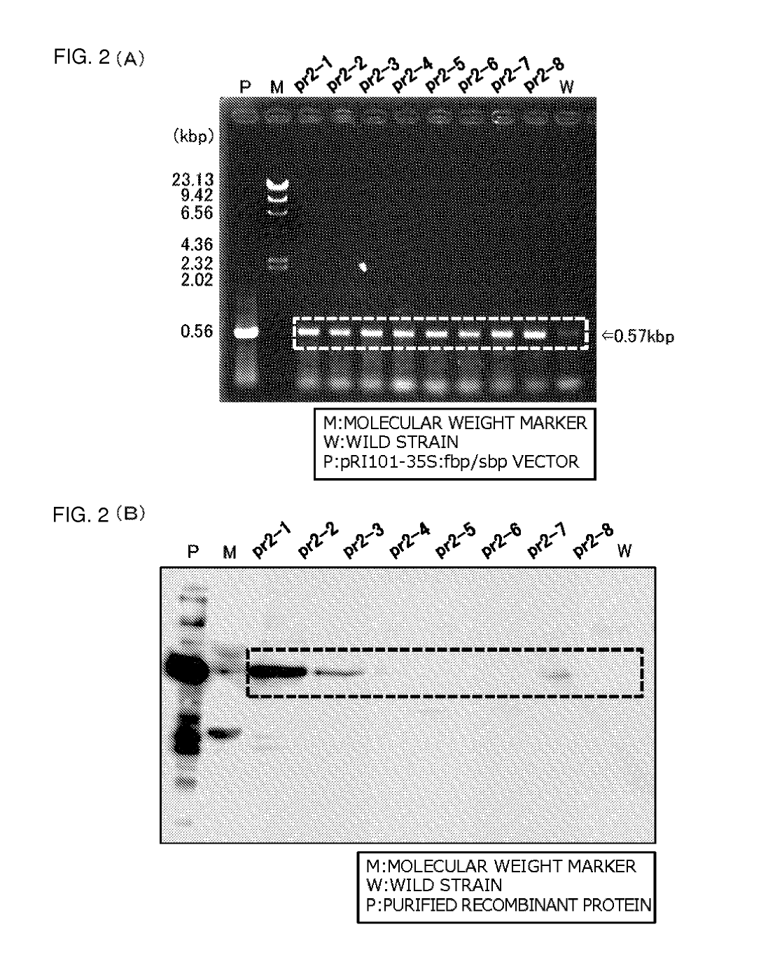

An antibiotic resistant strain, thus appearing, was suspended in a CM medium, and was plated on a selection medium. Colonies appearing one week after were subjected to streak culture, and were further subjected to selection. The PCR was carried out with respect to each of colonies obtained by selection, and a signal was recognized at a position of 0.57 kbp, as illustrated in FIG. 2(A). This indicates that in a nuclear genome, a base sequence that encodes a protein having FBPase/SBPase activities was introduced to 8 lines of transformants. The 8 lines of transformants thus obtained were named as PR2-1 to PR2-8, respectively, and the Western blotting analysis was performed.

As a result, as illustrated in FIG. 2(B), at least three lines of PR2-1, PR2-2, and PR2-7, a signal of 40 kDa, indicating a protein having activities of FBPase/SBPase derived from cyanobacteria, was detected.

### Example 2

### Comparison of Growth Between Transformed Strain and Wild Strain

Gene introduction with respect to Euglena was performed by the method described above as to the embodiments.

Conditions for preculture of Euglena were set to be identical to those in Example 1 except for the conditions of continuous irradiation for five days.

As the linear gene fragment, two types of linear gene fragments were used, which are a binary vector pBI121-35S for plants (produced by Takara Bio Inc.), and the binary vector pRI101-35S, which was used in Example 1 as well. A gene that encodes a protein having FBPase/SBPase activities (fbp/sbp, a gene including a base sequence represented by SEQ ID NO. 1 cloned by the inventors of the present invention) was inserted into multicloning sites of these binary vectors, while a chloroplast transit peptide rbcS-TP (acquired from Mr. Sugita in Nagoya University) was inserted in the upstream of the translation start sites thereof, whereby a vector pBI121-35S:fbp/sbp and a vector pRI101-35S:fbp/sbp were produced.

As to each of these vectors, a region interposed between LB and RB was amplified by PCR, whereby a linear gene fragment was obtained, and thereafter, under the same conditions as those in Example 1, gene introduction was carried out with respect to Euglena by the particle gun method.

After the introduction, selection was carried out through the same procedure as that in Example 1, and from the obtained drug-resistant strains, three transformed strains were obtained. Among the three strains thus obtained, one transformed strain (EpFS-1) was subcultured in a CM medium 1 L from which an antibiotic was withdrawn, at the same number of cells as that of the wild strain, so that an experiment for comparison of growth as described below was carried out.

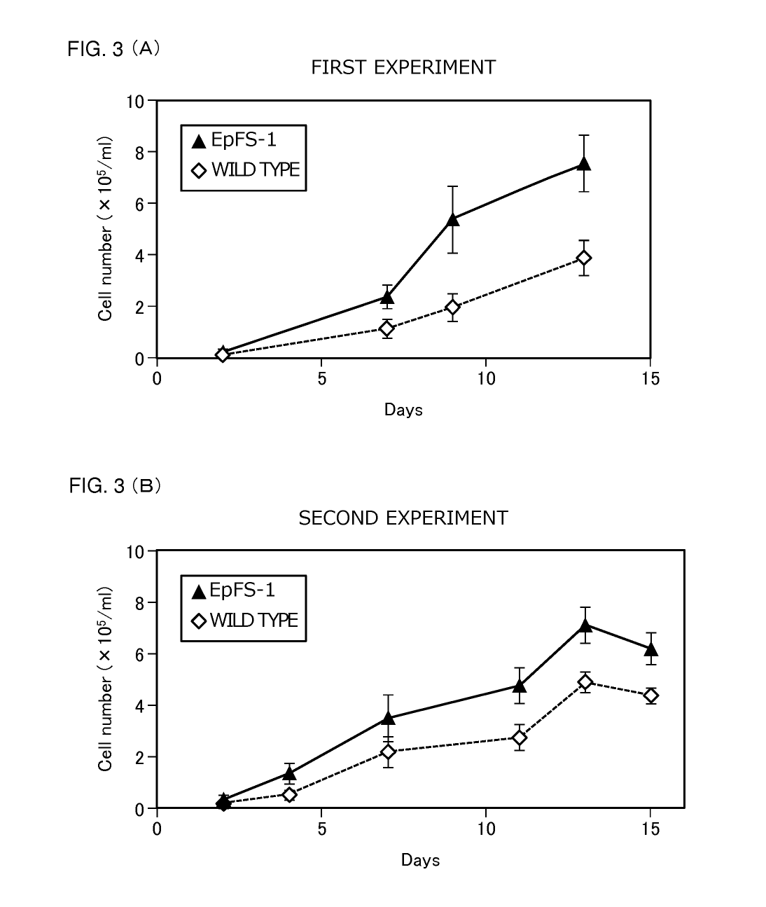

An experiment in which the transformed strain (EpFS-1) and the wild strain were subcultured in a CM medium (1 L) containing no antibiotic at the same number of cells was performed twice (the first experiment, and the second experiment illustrated in FIG. 3). These were sampled with time, the numbers of cells were measured, the cell sizes were observed, and further, the chlorophyll amounts thereof were measured. Besides, using cells in the stationary phase (day 14 after inoculation), and using an oxygen electrode, photosynthetic activity and respiratory activity were measured.

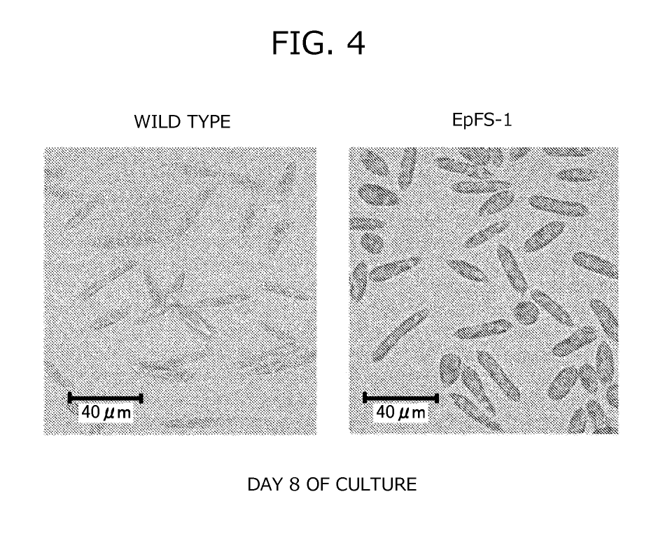

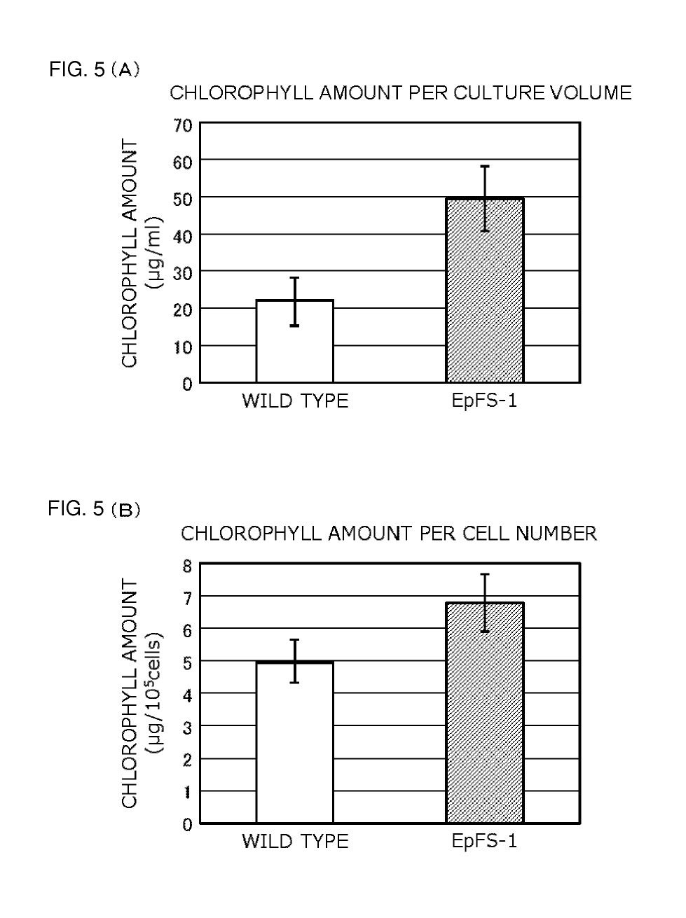

As a result, as compared with the wild strain, the cells of the transformed strain (EpFS-1) were larger as illustrated in FIG. 4, and the number of cells in the stationary phase was about 1.4 times that of the wild strain, as illustrated in FIG. 3. Besides, as illustrated in FIG. 5, the chlorophyll amounts per volume and per cell number of the transformed strain (EpFS-1) tended to increase to about 1.5 times, as compared with the wild strain.

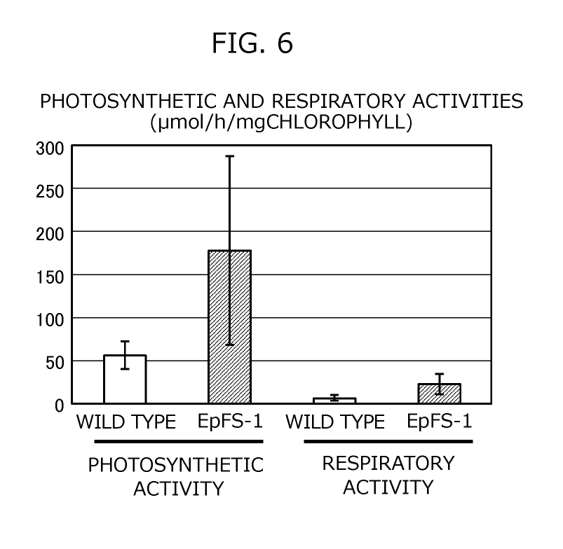

Still further, as illustrated in FIG. 6, which is to be mentioned below regarding Example 3, both of photosynthetic activity and respiratory activity of the transformed strain (EpFS-1) tended to be high, as compared with the wild strain.

### (Comparison of Average Particle Size and Cell Volume Between Wild Strain and Transformed Strain)

In FIG. 4, photographs showing cell sizes of the wild strain and the transformed strain (EpFS-1) are presented in a contrasting manner. In order to compare the cell size not only visually but also quantitatively, the following culture was performed, so that the cell sizes of these strains were compared.

Culture conditions were as follows.

The transformed strain (EpFS-1) obtained by gene introduction in Example 2 and the wild strain were cultured for six days in a CM medium (pH 5.5), in 50 mL test tubes, at a water temperature of 29° C., under the light conditions of continuous irradiation for 24 hours (200 μmol/m²/s), under the aeration of air alone, and under the aeration of a gas mixture of air and 5% CO₂, at a flow rate of 50 ml/min.

Euglena cells at day 6 of culture were sampled, and the average particle sizes thereof were measured with a CDA-1000 (Sysmex Corporation). The volumes thereof were calculated by substituting the average particle sizes as diameters into 4/3 πr³. The results are shown in Table 1.

TABLE 1

| | | Average particle | |
|---|---|---|---|
| | | size (μm) | Volume (μm³) |
| Air aeration | Wild strain | 11.6 | 263.1 π |
| condition | EpFS-1 | 15.3 | 593.8 π |
| CO₂ aeration | Wild strain | 13.6 | 421.3 π |
| condition | EpFS-1 | 14.2 | 472.4 π |

As indicated by the results shown in Table 1, the transformed strain (EpFS-1) had a greater average particle size than the wild strain, in both cases of the aeration of air alone, and the aeration of the mixture gas of air and 5% CO₂. This therefore quantitatively indicates that the volume of the transformed strain, which is calculated from the average particle size, was greater than the wild strain, too.

### (Comparison of Carbohydrate Content in Culture Solution Between Wild Strain and Transformed Strain)

Further, the wild strain and the transformed strain (EpFS-1) were compared regarding whether carbohydrate contents in culture solutions for Euglena were different between the wild strain and the transformed strain (EpFS-1).

The wild strain and the transformed strain (EpFS-1) of Euglena were cultured for 6 days through the same procedure as that for (Comparison of average particle size and cell volume between wild strain and transformed strain).

The Euglena culture solutions at day 6 of culture were collected, and were heated at 95° C. for one hour. After heating, the same was centrifuged (at 4,000 rpm for 5 minutes), and only the upper layer was collected. As the upper layer did not contain Euglena cells, Euglena cells were removed by collecting only the upper layer in this way. Next, regarding the collected upper layer, a carbohydrate content was quantitatively determined by the phenol-sulfuric acid method. This carbohydrate is sticky polysaccharide that Euglena secretes to outside the cells, and is called mucilage.

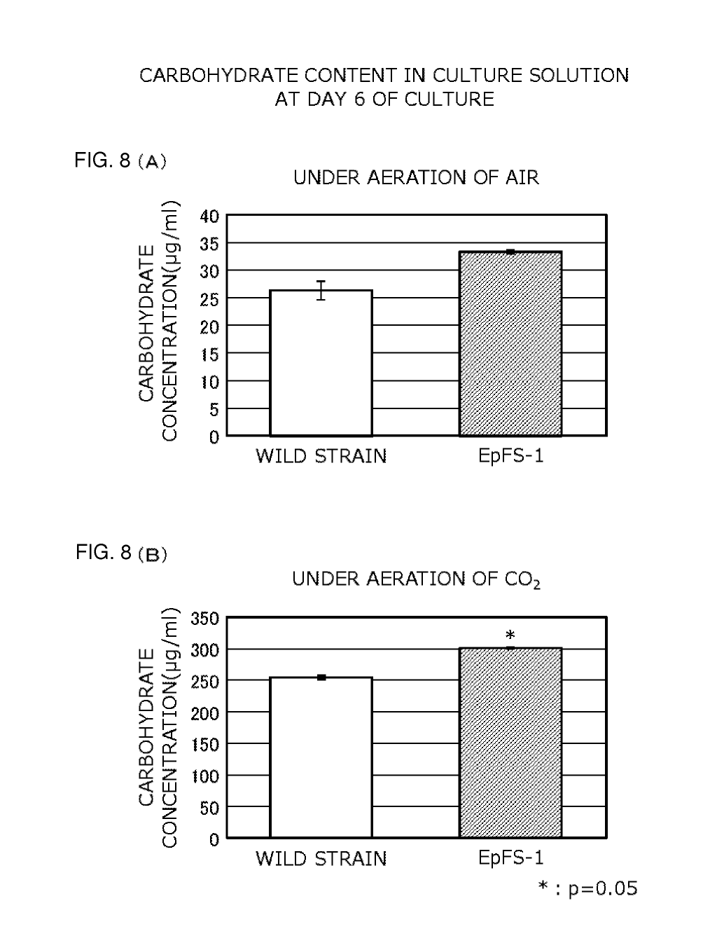

The results of quantitative determination are illustrated in FIG. 8.

The amounts of carbohydrate contained in the media in which the wild strain and the transformed strain (EpFS-1) were cultured were compared, and it was found that the medium in which the transformed strain (EpFS-1) was cultured contained more carbohydrate than the other medium, which indicates that the transformed strain (EpFS-1) secreted more mucilage than the wild strain.

### Example 3

### Culture after Gene Introduction

After gene introduction was carried out in Example 2, the wild strain and the transformed strain (EpFS-1) of Example 2 were subcultured, with the same numbers of cells, in a Cramer-Myers medium (hereinafter referred to as a “CM medium”, (NH₄)₂HPO₄: 1.0 g/L, KH₂PO₄: 1.0 g/L, MgSO₄.7H₂O: 0.2 g/L, CaCl₂.2H₂O: 0.02 g/L, citric acid 3Na.2H₂O: 0.8 g/L, Fe₂(SO₂)₃.7H₂O: 3 mg/L, MnCl₂.4H₂O: 1.8 mg/L, CoSO₄.7H₂O: 1.5 mg/L, ZnSO₄.7H₂O: 0.4 mg/L, Na₂MoO₄.2H₂O: 0.2 mg/L, CuSO₄.5H₂O: 0.02 g/L, thiamine hydrochloride (vitamin B₁): 0.1 mg/L, cyanocobalamin (vitamin B₁₂): 0.0005 mg/L, (pH3.5)).

These culture solutions were sampled with time, and the numbers of cells thereof were counted. Further, using an oxygen electrode, photosynthetic activity and respiratory activity were measured.

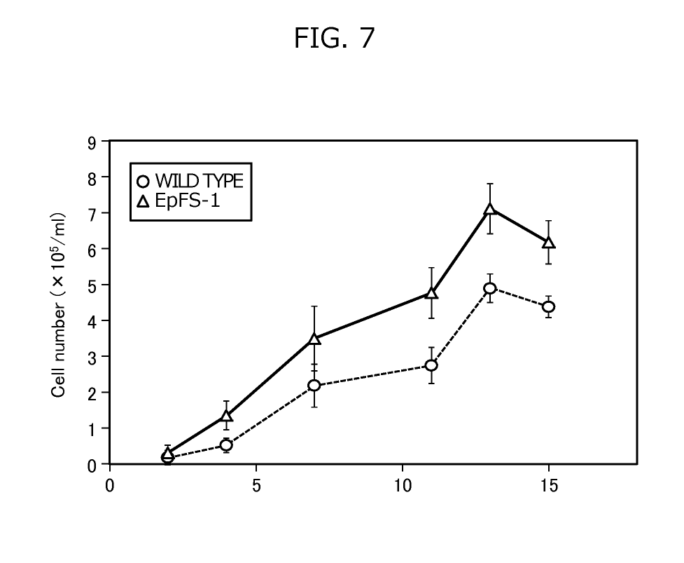

As illustrated in FIG. 7, at day 13 from the start of culture, the cell concentration of the transformed strain (EpFS-1) reached 1.4 times that of the wild strain.

Further, as illustrated in FIG. 6, the transformed strain (EpFS-1) exhibited higher photosynthetic activity and respiratory activity than those of the wild strain.

The above-described results suggest that the base sequence that encodes a protein having FBPase/SBPase activities, which was introduced to the transformed strain (EpFS-1), functioned in the Euglena cells into which the base sequence was introduced, and enhanced the photosynthetic activity of Euglena, thereby increasing the proliferation rate of the Euglena cells of the transformed strain, as compared with the wild strain.

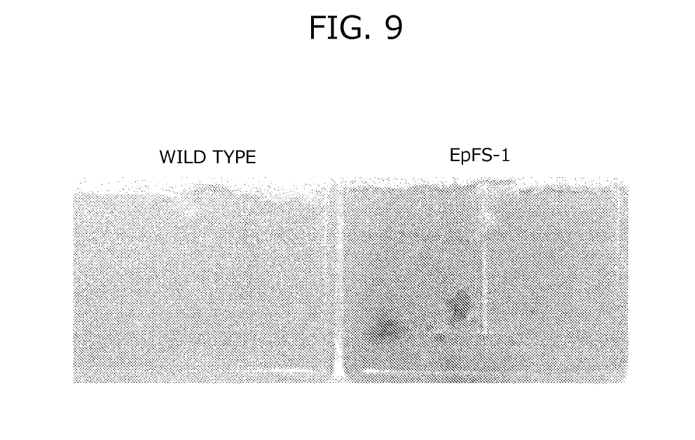

Still further, as illustrated in FIG. 9, mucilage was observed as dark-color floating substances, only in the culture solution of the transformed strain (EpFS-1) illustrated in the right-side part of the drawing. Based on this, it can be considered that, with the increase of photosynthesis, the produced amount of carbohydrate as a product of photosynthesis increased in the transformed strain (EpFS-1), and apart of the same was secreted to outside the cells, which resulted in the accumulation of mucilage as illustrated in FIG. 9.

1. A method for introducing a gene into Euglena, wherein a DNA fragment comprising a base sequence that encodes a protein is introduced to a Euglena cell.

2. The method for introducing a gene into Euglena according to claim 1, the method comprising:
producing a binary vector containing the DNA fragment;
obtaining a linear gene fragment that includes a T-DNA region including the DNA fragment in the binary vector; and
introducing the linear gene fragment into the Euglena cell by a direct gene introduction step.

3. The method for introducing a gene into Euglena according to claim 2,
wherein the direct gene introduction step further comprises:
coating a microcarrier with the linear gene fragment; and
injecting the microcarrier coated with the linear gene fragment to the Euglena cell with a particle gun.

4. The method for introducing a gene into Euglena according to claim 3,
wherein the DNA fragment is a DNA fragment comprising a base sequence encoding a protein having activities of fructose-1,6-bisphosphatase and sedoheptulose-1,7-bisphosphatase derived from cyanobacteria.

5. The method for introducing a gene into Euglena according to claim 3,
wherein the microcarrier is a gold microparticle having a diameter of 0.26 μm or less.

6. The method for introducing a gene into Euglena according to claim 4,
wherein a transformed strain of Euglena is obtained, characterized by improved number of proliferated cells, cell size, chlorophyll amount, photosynthetic activity, and respiratory activity.

7. The method for introducing a gene into Euglena according to claim 4,
wherein the Euglena is Euglena gracilis.

8. The method for introducing a gene into Euglena according to claim 4,
wherein the protein has an amino acid sequence indicated in (a) or (b) below:
(a) an amino acid sequence corresponding to amino acid residues 1 to 356 of the amino acid sequence represented by SEQ ID NO. 2;
(b) an amino acid sequence that is identical to the amino acid sequence of (a) with a part thereof is deleted, substituted or added, having fructose-1,6-bisphosphatase and sedoheptulose-1,7-bisphosphatase activities.

9. The method for introducing a gene into Euglena according to claim 4,
wherein the base sequence is a base sequence indicated in (c) or (d) below:
(c) a base sequence corresponding to nucleotides 181 to 1251 of the base sequence represented by SEQ ID NO. 1;
(d) a base sequence that is identical to the base sequence of (c) with a part thereof deleted, substituted, or added, and the base sequence encodes a protein having fructose-1,6-bisphosphatase and sedoheptulose-1,7-bisphosphatase activities.

10. A transformant of Euglena, obtained by introducing, into Euglena, a gene that encodes a protein having activities of fructose-1,6-bisphosphatase and sedoheptulose-1,7-bisphosphatase derived from cyanobacteria.

11. The transformant of Euglena according to claim 10,
wherein the Euglena is Euglena gracilis.

12. The transformant of Euglena according to claim 11,
wherein the gene is a gene that encodes a protein of (a) or (b) below:
(a) a protein having an amino acid sequence corresponding to amino acid residues 1 to 356 of the amino acid sequence represented by SEQ ID NO. 2;
(b) a protein having an amino acid sequence corresponding to amino acid residues 1 to 356 of the amino acid sequence represented by SEQ ID NO: 2, with one or more amino acid substitution(s), deletion(s), insertion(s), and/or addition(s) in the amino acid sequence corresponding to amino acid residues 1 to 356 of the amino acid sequence represented by SEQ ID NO. 2, and having fructose-1,6-bisphosphatase and sedoheptulose-1,7-bisphosphatase activities.

13. The transformant of Euglena according to claim 11,
wherein the gene has a base sequence of (c) or (d) below:
(c) a base sequence corresponding to nucleotides 181 to 1251 of the base sequence represented by SEQ ID NO. 1;
(d) a base sequence corresponding to nucleotides 181 to 1251 of the base sequence represented by SEQ ID NO. 1, with one or more nucleotide base substitution(s), deletion(s), insertion(s), and/or addition(s) in the base sequence corresponding to nucleotides 181 to 1251 of the base sequence represented by SEQ ID NO. 1, and that encodes a protein having fructose-1,6-bisphosphatase and sedoheptulose-1,7-bisphosphatase activities.

14. The transformant of Euglena according to claim 11,
wherein the gene is introduced to a nuclear genome and/or a chloroplast genome of the Euglena.
